(19) United States
(12) Patent Application Publication
(10) Pub. No.: US 2022/0362064 A1
(43) Pub. Date: Nov. 17, 2022

(54) ADHESIVE WOUND DRESSING AND METHOD OF APPLICATION THEREOF FOR DRESSING LACERATIONS

(71) Applicant: J&C Medical Pty Ltd, Sydney (AU)

(72) Inventor: Jin Choong, Sydney (AU)

(21) Appl. No.: 17/662,904

(22) Filed: May 11, 2022

(30) Foreign Application Priority Data
May 12, 2021 (AU) 2021901402

(51) Int. Cl. A61F 13/10; A61F 13/00

(52) U.S. Cl. CPC A61F 13/105; A61F 13/00063; A61F 2013/00544

(57) ABSTRACT

An adhesive wound dressing has a body forming a first pair of limbs and a second pair of cross limbs. The first pair of limbs meet the second pair of cross limbs substantially orthogonally at a central juncture. The limbs each have an adhesive surface on a contact side of the body. The dressing has at least one peel-off sheet covering adhesive surfaces of the limbs and a non-adhering central padding covering the contact side of the central juncture. The dressing may be used for dressing lacerations including digit lacerations such as distal or interdigital lacerations.

### FIELD OF THE INVENTION

This invention relates generally to adhesive wound dressing and method of application thereof for dressing lacerations.

SUMMARY OF THE DISCLOSURE

There is provided herein an adhesive wound dressing to address deficiencies of conventional dressings, bandages and the like for dressing digits, including distal and interdigital lacerations. The present adhesive wound dressing may be suitable for lacerations where sutures are not possible (such as completely sliced off nail beds).

Specifically, the present adhesive wound dressing comprises a body forming a first pair of limbs and a second pair of cross limbs. The first pair of limbs meet the second pair of cross limbs substantially orthogonally at a central juncture. The limbs each have an adhesive surface on a contact side of the body.

The dressing further comprises at least one peel-off sheet covering adhesive surfaces of the limbs and a non-adhering central padding covering the contact side of the central juncture.

As such, for dressing a distal end laceration of a digit, the first limbs may be adhered along and over the end of the digit whereas the cross limbs may wrap around sides of the digit. In a preferred embodiment the cross limbs have curvature in the same direction so that, in this application, the cross limbs may curve back along the length of the digit in from the distal end thereof. In embodiments, the cross limbs curve to same extent as one of the first limbs for substantial coextensive side adhesion.

For dressing interdigital lacerations, the first limbs may be adhered substantially parallel and over between adjacent digits whereas the cross limbs adhere over respective adjacent digits. Curvature of the cross limbs may allow for proper adhesion on the top or bottom surface of the hand or foot.

For skin lacerations, the cross limbs may pull the laceration closed. Treating a skin laceration may comprise pulling a laceration longitudinally with opposing fingers to close the laceration whereafter the cross limbs may be applied to hold the laceration closed. Thereafter, the fingers may be removed whereafter the first limbs are adhered.

Exemplary dimensions are provided herein specifically suited for fingers and toes.

Embodiments of the central padding provided herein comprise combinations of layers and/or additives to promote healing, preventing damage to the skin healing layer and/minimises complication of wound infection.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a perspective view of an adhesive wound dressing in accordance with an embodiment;

FIG. 2 shows a side elevation view of the dressing of FIG. 1;

FIG. 3 illustrates utilisation of the dressing for dressing fingers;

FIG. 4 illustrates utilisation of the dressing for dressing skin lacerations;

FIG. 5 shows a plan view of the dressing having dimensions suited for the fingers;

FIG. 6 shows a plan view of the dressing having dimensions suited for the big toe; and

FIG. 7 shows a plan view of the dressing having dimensions suited for the smaller toes.

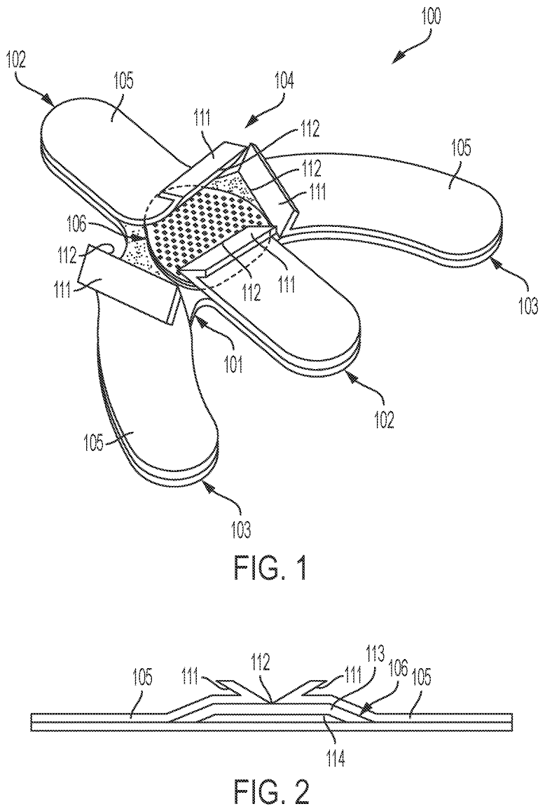

### DESCRIPTION OF EMBODIMENTS

An adhesive wound dressing 100 comprises a body 101 comprising a first pair of limbs 102 and a second pair of cross limbs 103. The first pair of limbs 102 meet the second pair of cross limbs 103 substantially orthogonally at a central juncture 104.

The limbs 102, 103 each have an adhesive surface on a contact side of the body 101 to adhere to the skin.

The dressing 100 comprises at least one peel-off sheet 105 covering adhesive surfaces of the limbs 102, 103.

The dressing 100 further comprises a non-adhering central padding 106 covering the contact side of the central juncture 104.

Referencing FIG. 5, the first pair of limbs 102 may be straight and in alignment and the second pair of cross limbs 103 may curve in the same direction.

As is further shown in FIG. 5, the cross limbs 103 may curve to an end of the body 101. Specifically, the distal ends 107 of the cross limbs 103 may be substantially collinear with distal ends 108 of one of the first limbs 102 (i.e. lie substantially along the imaginary line 109).

The limbs 102, 103 may generally be of uniform width along respective lengths thereof. In other words, each limb 102, 103 may have a central section 109 between the central juncture 104 and a distal end 107, 108 thereof which are uniform in width.

The central padding 106 may have a dimension greater than a maximum width of the limbs 102, 103. Specifically, as is further shown in FIG. 5, the limbs 102, 103 may meet at rounded shoulders 110 which increase the available surface area of the juncture 104 to allow for central padding 106 having a dimension being greater than a maximum width of the limbs 102, 103. Specifically, referencing FIG. 5, the central padding 106 may comprise a width of 3 cm with respect to a longitudinal axis of the first pair of limbs 102 wherein the first pair of limbs 102 may have a maximum width of 2 cm. Furthermore, the central padding 106 may comprise a width of 3 cm with respect to a longitudinal axis of the cross limbs 103 whereas the cross limbs 103 may comprise a width of 1.8 cm.

The at least one peel-off sheet 105 may comprise a peel-off sheet 105 for each limb 102, 103. Each peel-off sheet 105 may comprise a pull tab 111. Referencing FIG. 2, fold lines 112 of the pull tabs 111 may meet.

Further referencing FIG. 2, the central padding 106 may comprise an outer mesh layer 113 and an absorbent layer 114 thereunderneath.

The padding 106 (especially the outer mesh layer 113) may comprise a topical antibiotic such as mupirocin. The outer mesh layer 113 may further comprise paraffin.

In embodiments, the outer mesh layer 113 may comprise medical grade honey.

The absorbent layer 114 may comprise cotton or similar absorbent material.

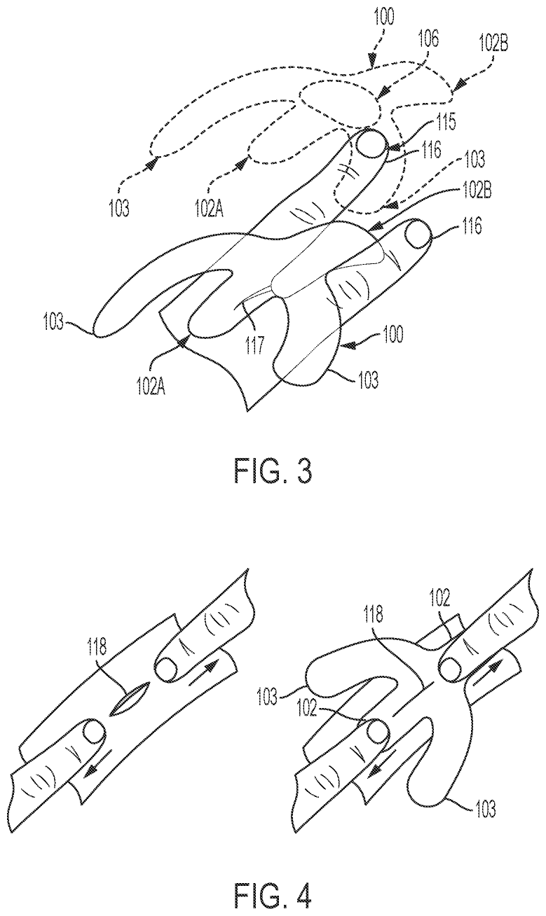

FIG. 3 illustrates the method of using the adhesive wound dressing 100 to dress a distal end laceration 115 (such as a cut or torn nail and/or skin) of a digit 116 (such as a finger or toe).

The method may comprise adhering the first pair of limbs along and over the digit 116 whereas the second pair of cross limbs 103 wrap around the digit 116 with the central padding locating over the laceration 115.

More specifically, limb 102A may be applied first (by removing the peel-off sheet 105 and adhering the adhesive surface thereof across the top of the digit 116) followed by limb 102B.

Thereafter cross limbs 103 may each be adhered in turn.

As can be appreciated, the curvature of the cross limbs 103 allow the cross limbs 103 to adhere back along the longitude of the digit 116 in from the distal end of the digit 116.

FIG. 3 further illustrates a method of using adhesive wound dressing 100 to dress an interdigital webbing laceration 117 wherein the first pair of limbs 102 are applied between the digits 116 substantially parallel thereto whereafter the cross limbs 103 adhere over or under respective adjacent digits 116.

The dressing 100 may be applied in the same sequence as described above.

FIG. 4 further illustrates the utilisation of the adhesive wound dressing 100 to dress a skin laceration 118 wherein the cross limbs 103 pull the skin laceration 118 closed.

More specifically, the laceration 118 may be pulled longitudinally as shown to pull the laceration 118 closed. Thereafter, the cross limbs 103 may be adhered first to bear orthogonally across the laceration 118 to hold the laceration closed. Thereafter, the first pair of limbs 102 may be adhered.

FIG. 5 shows exemplary dimensions of the dressing 100 suited for fingers and webbing therebetween. In accordance with this embodiment, the first limbs 102 may comprise a width of approximately 2 cm whereas the cross limbs 103 may comprise a width of approximately 1.8 cm. The body 101 may comprise a length of approximately 8 cm along the first pair of limbs 102. The central padding 106 may comprise a length of approximately 3 cm and a width of approximately 2 cm.

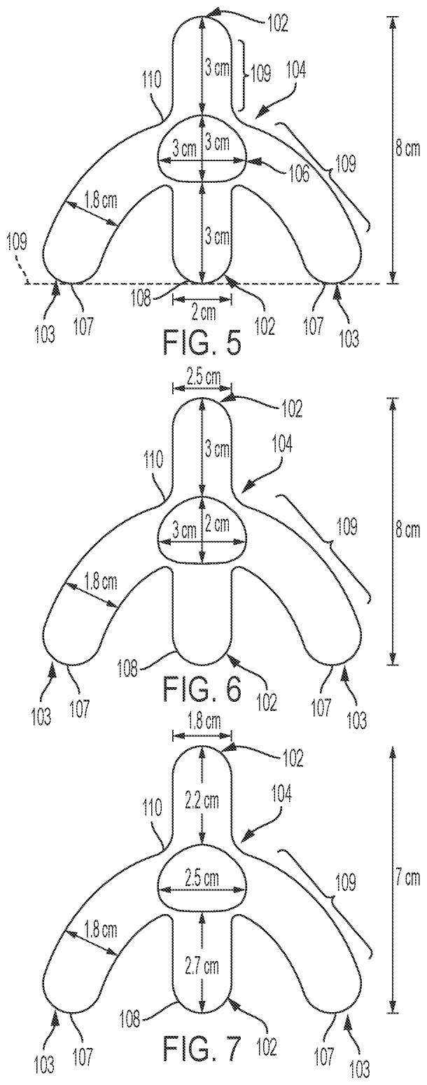

FIG. 6 shows an embodiment of the dressing 100 suited for the big toe which may have substantially the same dimensions as the embodiment shown in FIG. 5 except that each limb 102 is 2.5 cm wide.

FIG. 7 shows an embodiment of the dressing 100 suited for the smaller toes wherein the first limbs 102 are narrower and shorter as compared to the embodiments of FIGS. 5 and 6. Specifically, the first limbs 102 may comprise a width of less than 2.5 cm (preferably approximately 1.8 cm) and a combined length of approximately 7 cm.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

It should be noted that the term “laceration” and derivatives thereof used herein should be construed broadly as being any type of skin wound.

1. An adhesive wound dressing comprising:
a body forming:
a first pair of limbs;
a second pair of cross limbs, wherein:
the first pair of limbs meet the second pair of cross limbs substantially orthogonally at a central juncture; and
the limbs each have an adhesive surface on a contact side of the body;
at least one peel-off sheet covering adhesive surfaces of the limbs; and
a non-adhering central padding covering the contact side of the central juncture.

2. The dressing as claimed in claim 1, wherein the first pair of limbs are straight and in alignment and the second pair of cross limbs curve in the same direction.

3. The dressing as claimed in claim 2, wherein the cross limbs curve substantially to an end extend of the body.

4. The dressing as claimed in claim 2, wherein distal ends of the cross limbs are substantially collinear with a distal end of one of the first arms.

5. The dressing as claimed in claim 1, wherein each limb has a section between the central juncture and distal end thereof which is uniform in width.

6. The dressing as claimed in claim 1, wherein the at least one peel-off sheet comprises a peel-off sheet for each limb.

7. The dressing as claimed in claim 6, wherein each sheet comprises a pull tab located at the central juncture.

8. The dressing as claimed in claim 7, wherein fold lines of opposite pull tabs meet.

9. The dressing as claimed in claim 1, wherein the central padding has a dimension greater than a maximum width of the limbs.

10. The dressing as claimed in claim 9, wherein the limbs meet at rounded shoulders which increase the available surface area of the juncture.

11. The dressing as claimed in claim 1, wherein the cross limbs comprise a width less than that of the first limbs.

12. The dressing as claimed in claim 1, wherein the body comprises a length of approximately 8 cm along the first limbs.

13. The dressing as claimed in claim 1, wherein the body comprises a length of approximately 7 cm along the first limbs.

14. The dressing as claimed in claim 1, wherein the first limbs comprise a width of approximately 2.5 cm.

15. The dressing as claimed in claim 1, wherein the first limbs comprise a width of less than 1.8 cm.

16. The dressing as claimed in claim 1, wherein the cross limbs comprise a width of less than 2 cm.

17. The dressing as claimed in claim 1, wherein the cross limbs comprise a width of approximately 1.8 cm.

18. The dressing as claimed in claim 1, wherein the central padding comprises an outer mesh layer and an absorbent layer thereunder.

19. The dressing as claimed in claim 18, wherein the outer mesh layer comprises a topical antibiotic.

20. The dressing as claimed in claim 19, wherein the topical antibiotic comprises mupirocin.

21. The dressing as claimed in claim 18, wherein the outer mesh layer comprises paraffin.

22. The dressing as claimed in claim 18, wherein the outer mesh layer comprises medical grade honey.

23. The dressing as claimed in claim 1, wherein the central padding comprises a topical antibiotic.

24. The dressing as claimed in claim 23, wherein the topical antibiotic comprises mupirocin.

25. A method of using the adhesive wound dressing as claimed in claim 1 to dress a distal end laceration of a digit, the method comprising adhering the first pair of limbs along and over the digit whereas the second pair of cross limbs wrap around the digit with the central padding locating over the laceration.

26. The method as claimed in claim 25, wherein first limbs are applied in turn followed by the cross limbs applied in turn.

27. A method of using the adhesive wound dressing as claimed in claim 1 to dress an inter-digital webbing laceration, the method comprising applying the first pair of limbs between the digits wherein the cross limbs adhere over or under respective adjacent digits.

28. The method as claimed in claim 27, wherein first limbs are applied in turn followed by the cross limbs applied in turn.

29. A method of using the adhesive wound dressing as claimed in claim 1 to dress a skin laceration, wherein the cross limbs pull the skin laceration closed.

30. The method as claimed in claim 29, further comprising pulling the laceration longitudinally to close the laceration, adhering the cross limbs across the laceration and then adhering the first pair of limbs.
